(19) United States
(12) Patent Application Publication
(10) Pub. No.: US 2014/0245817 A1
(43) Pub. Date: Sep. 4, 2014

(54) AIRBORNE PARTICLE COLLECTION DEVICE APPLICATION

(71) Applicants: H. Johnny Gainey, JR., Sunset Beach, NC (US); Randy Hardee, Sunset Beach, NC (US)

(72) Inventors: H. Johnny Gainey, JR., Sunset Beach, NC (US); Randy Hardee, Sunset Beach, NC (US)

(21) Appl. No.: 14/172,302

(22) Filed: Feb. 4, 2014

Related U.S. Application Data
(60) Provisional application No. 61/760,220, filed on Feb. 4, 2013.

(51) Int. Cl. G01N 33/00; G01N 33/483

(52) U.S. Cl. CPC G01N 33/0009; G01N 33/483
USPC 73/31.02

(57) ABSTRACT

The present application is directed to a computer-based application and corresponding method and apparatus of analysing air quality inside a motor vehicle. One example may provide conducting an air sampling procedure of air inside the motor vehicle to obtain samples of air from inside the vehicle for comparison to a baseline or air outside the vehicle. The operation may also include identifying an existence of one or more substances within that air sampled inside the motor vehicle via one or more sensors and calculating densities of the one or more identified substances via a processor. The air may contain one or more of the substances in a particular quantity of interest to the user. The calculated densities may then be compared to corresponding threshold density values stored in memory, and an electronic report identifying the findings of the calculations may be generated to present to the user.

### I. RELATED APPLICATIONS

This application claims priority to U.S. Provisional Patent Application No. 61/760,220, filed on Feb. 4, 2013 and entitled, “Airborne Particle Collection Device Application”. The entire contents of which are herein incorporated by reference.

### II. FIELD OF INVENTION

The present invention relates to a computer application that operates to detect and report airborne particle and microorganism collection performed by a portable airborne particle system.

### III. BACKGROUND OF THE INVENTION

Conventional airborne particle collection, sampling, and screening kits, devices, and systems have been used for a variety of testing purposes. However, most systems are not highly portable and are not necessarily optimal for collecting airborne particles and microorganisms, and which utilizes an adapter configured for use with a commercially-available vacuum source. An adapter that is used to generate a constant air flow pull volume rate of a selectively desired number of liters per minute, irrespective of selected vacuum source, in a manner which is quick, easy, and efficient is desired.

Also, a computer-based application that receives the sensor data, the measured particles and other data and which generates a result that may be transmitted to a user on-the-fly is also desired. The application may provide users accurate information and warnings regarding the density or existence of certain molds (moulds), microorganisms and particles identified during a screening phase.

### IV. SUMMARY OF THE INVENTION

Example embodiments of the present application disclose hardware, software and/or operations and procedures configured to analyse air quality inside a motor vehicle, identify the existence of one or more substances, such as molds (moulds), microorganisms, and particles, calculate densities of the identified substances, compare the densities to threshold density values and generate an electronic report identifying the findings of the calculations and notify the user via a warning message if any indicators have exceeded the threshold density values.

### V. BRIEF DESCRIPTION OF THE DRAWING(S)

FIG. 1 illustrates an example graphical user interface (GUI) illustrating the user accessible test report information, in accordance with example embodiments.

FIG. 2 illustrates an example graphical user interface (GUI) illustrating user input options to setup a test report, in accordance with example embodiments.

FIG. 3A illustrates another example graphical user interface (GUI) illustrating user input options to setup a test report, in accordance with example embodiments.

FIG. 3B illustrates yet another example graphical user interface (GUI) illustrating user input options to setup a test report, in accordance with example embodiments.

FIG. 3C illustrates still another example graphical user interface (GUI) illustrating test report summaries, in accordance with example embodiments.

FIG. 3D illustrates still yet another example graphical user interface (GUI) illustrating test report results generated by a computer application, in accordance with example embodiments.

FIG. 4 illustrates an airflow measurement circuit, in accordance with example embodiments.

FIG. 5 illustrates an example electronics enclosure of a timer mechanism, according to example embodiments.

FIG. 6 illustrates a flow diagram of an example method according to an example embodiment of the present application.

FIG. 7 illustrates an example network entity device configured to store instructions, software, and corresponding hardware for executing the same, according to example embodiments of the present application.

### VI. DETAILED DESCRIPTION OF THE EMBODIMENT(S)

It will be readily understood that the components of the present application, as generally described and illustrated in the figures herein, may be arranged and designed in a wide variety of different configurations. Thus, the following detailed description of the embodiments of a method, apparatus, computer application and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments. Additionally, any references to a computer or electronics device may be directed to a computer, laptop, mobile, wireless or cellular phone, a PDA, a tablet, a client a server or any device that contains a processor and/or memory, whether that processor or memory performs a function related to an embodiment of the invention.

According to example embodiments, an air flow control mechanism is adapted to control the air flow volume as air is pulled through an air flow control and measurement system via a vacuum source. The air flow control mechanism is adapted to maintain a constant air flow pull volume rate of a selectively desired number of liters, i.e., 5 liters, 10 liters, 15 liters, or 20 liters per minute. The air flow control mechanism may be mounted to a vacuum nozzle adapter or may be included as an integral component of a vacuum source.

In accordance with yet another embodiment, the system of the present application may include sensors which communicate with a control unit or control processor (i.e., computer) to cause a sample to be collected in response to an event that is detected by the sensors. Such a system might be equipped with a mold (mould) spore monitor, and when levels of mold (mould) spores achieve a predetermined level, a controller can be programmed via the computer to initiate a sampling event, such as, to actuate a vacuum source to collect samples on the collection surface of a sample cassette for later analysis in response to the sensor readings. Such sensors can be used to measure relevant environmental factors. Based on the detection of a specific environmental factor by such a sensor, or in accordance with a sampling protocol programmed into the control, various functions may be executed by the control. Such functions may include but are not limited to a recording of generation of the environmental conditions at the time of detection, operation control of any system components whose performance depends on measured environmental parameters, manipulation of a programmed sampling protocol based on measured environmental factors, and production of an alert signal to notify an operator or user of an important change in the environmental conditions as determined by programmed control parameters. Also, a timer may be included to provide a timing signal to the control. The system of the present application is envisioned to be made commercially available as a kit, wherein the kit comprises a package for housing a number of sample cassettes, a first length of flexible tubing, an adapter, a second length of flexible tubing, a fitting, a vacuum nozzle adapter, a computer accessible interface and/or a mobile station or computing device interface for easy adaptation to a computer processing device, and an instruction leaflet.

In accordance to yet another embodiment, the system of the present application may include an electronic microscope which communicates with a control unit or control processor. The electronic microscope comprises an image collection means for obtaining images of collected and/or screened samples. The obtained images may be remotely transmitted wirelessly in real time to a network, such as a network of a laboratory sample testing facility. Thus, the instant embodiment allows for a field technician to immediately transmit images of collected and/or screened samples to the designated laboratory.

In accordance to still another embodiment, the system of the present invention comprises means for performing polymerase chain reaction deoxyribonucleic Acid (PCR DNA) analysis and/or testing. PCR DNA analysis and/or testing is characterized as a specialty test procedure and performed only upon specific request, and when so requested, is further identified and logged into the system platform of the present invention.

In accordance to still another embodiment, the system of the present invention comprises means for performing Environmental Relative Moldiness Index (ERMI) testing services. The ERMI testing is conducted by analyzing at least one sample of dust from a residential or commercial dwelling, or other interior or enclosure. The sample is analyzed using mold-specific quantitative polymerase chain reaction (MSQPCR), wherein MSQPCR is a highly specific DNA-based method for quantifying mold species. A ratio development algorithm is provided to calculate a ratio of water damage-related species to common indoor molds. A resulting ratio or score may be referred to as the Environmental Relative Moldiness Index or ERMI.

According to example embodiments, an airborne particle and microorganism collection system, “system”, may include an air particle and microorganism sample cassette for collecting airborne particles and microorganisms, the sample cassette may have an air intake and a media defining a collection surface to which air particles and microorganisms are collected. The system is designed and configured for the collection of air particle and microorganism samples, some of the air particles and microorganisms may be allergenic, pathogenic, and/or toxigenic and include bacteria, viruses, bacterial spores, mold (mould) spores, fungi, cellulose fibers, fiberglass particulates, insect fragments, and pollen. A detailed example of the system is included in U.S. application Ser. No. 13/077,243, entitled “Airborne particle and microorganism collection system” filed on Mar. 31, 2011, which is incorporated by reference in its entirety.

In operation, the air flow control mechanism of the adapter provides unanticipated and nonobvious functional features and advantages. The air flow control mechanism is adapted to control the air flow volume as air is pulled through the system via the vacuum source, the air flow control mechanism is adapted and configured to maintain a constant air flow pull volume rate of 5 liters per minute when interconnected with remaining elements comprising the system of the present application and when using any commercially-available vacuum source. It is envisioned the air flow control mechanism may be designed, configured, and manufactured in a number of models so as to provide a constant air flow pull volume rate according to consumer need and preference. Also, the computing device may be configured to automatically change the flow pull volume rate or other control functions via a user interface or via an automatic sensor or detection mechanism.

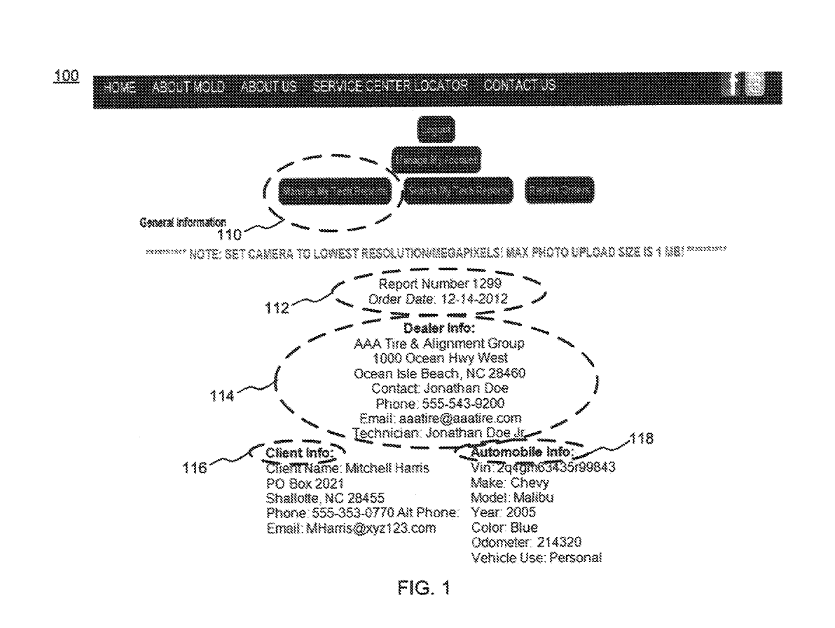

FIG. 1 illustrates an example test report user interface according to example embodiments. Referring to FIG. 1, the GUI 100 includes various features and functions used to generate, update, and produce an air quality report. A user may begin by logging into the report system via a web portal or online resource application on his or her computing device or smartphone. The user may access previous reports via an access report access option 110 on the GUI. The user may also upload photos, identify a report number 112 while the report is setup or after it has been generated, identify a dealer 114 who processed the report and performed the screening via the vacuum device, identify his or her client information 116 and the automobile information 118. Alternatively, the dealer may be the party responsible for identifying and accessing the generated reports.

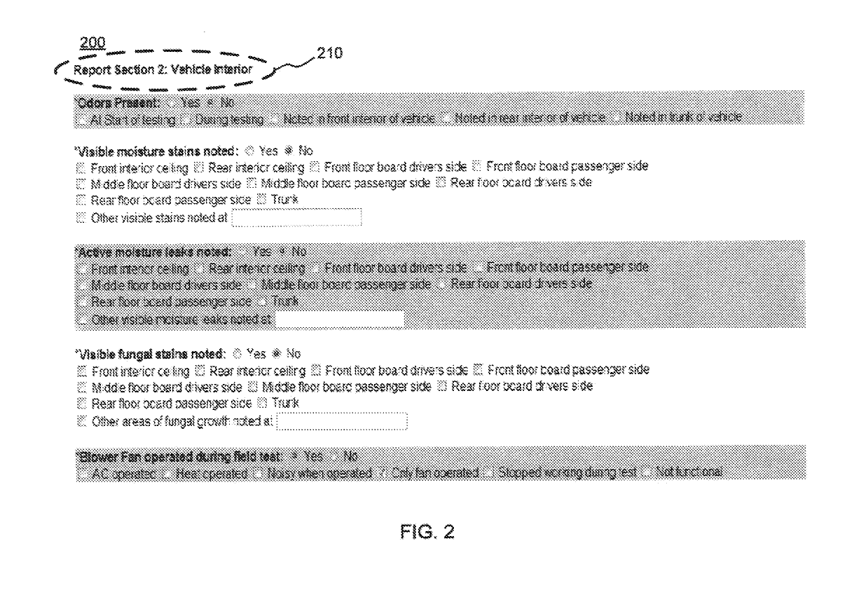

FIG. 2 illustrates another GUI 200 of a user setup option for the vehicle air diagnostic test. Referring to FIG. 2, a report section 210 may include various options to be selected for describing the vehicle, such as noticeable moisture presence and corresponding locations, active moisture leaks, visible fungus stations and a status of the blower fan. This information can be used to setup a proper vacuum test, interval time, volume rate and other test parameters which may be setup automatically via the computer application depending on the parameters input by the user during the test setup. For example, if the user indicated that the fungus was visible or is visible in more than a predefined number of locations (e.g., more than 2, 3 or 4, etc.) then the air diagnostic test may be conducted at a maximum air flow rate, for a maximum interval or vice versa depending on the likelihood of success of mold (mould) or particle detection. Predefined thresholds may be setup to identify user input parameters, specified mold (mould), allergen, spore or particle levels measured from air inside the vehicle and to conduct a corresponding set of test parameters (e.g., interval time, air flow rate, detection sensor initialization, particle concentration levels, etc.).

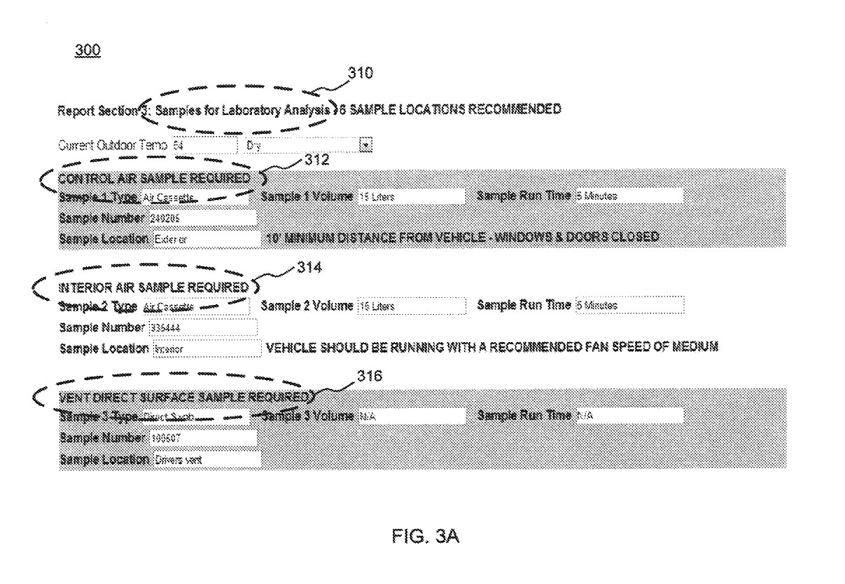

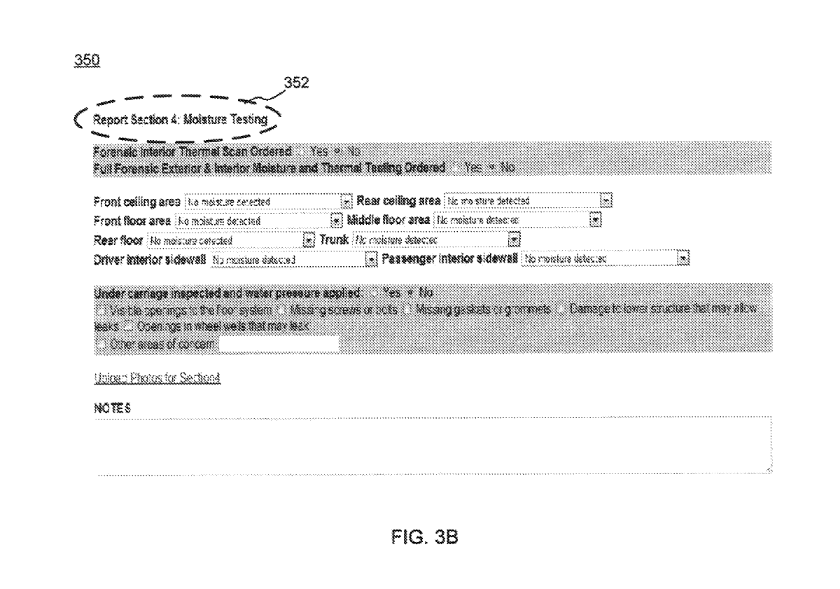

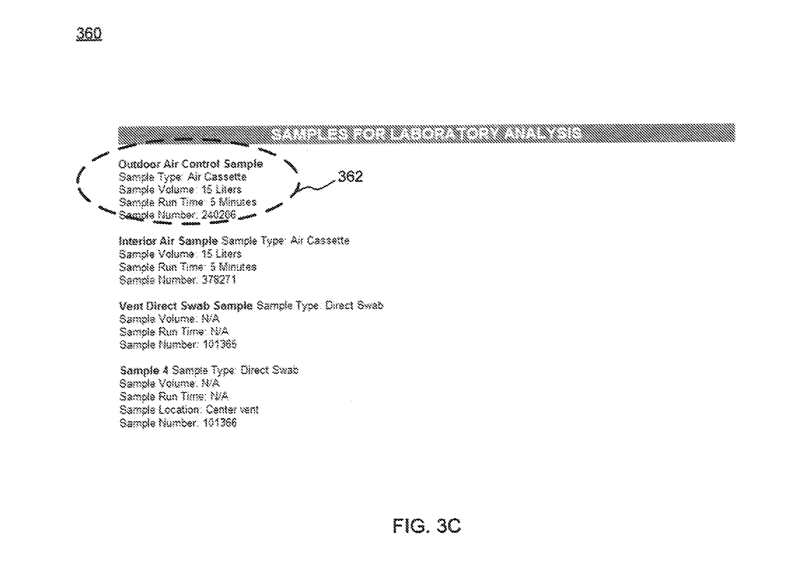

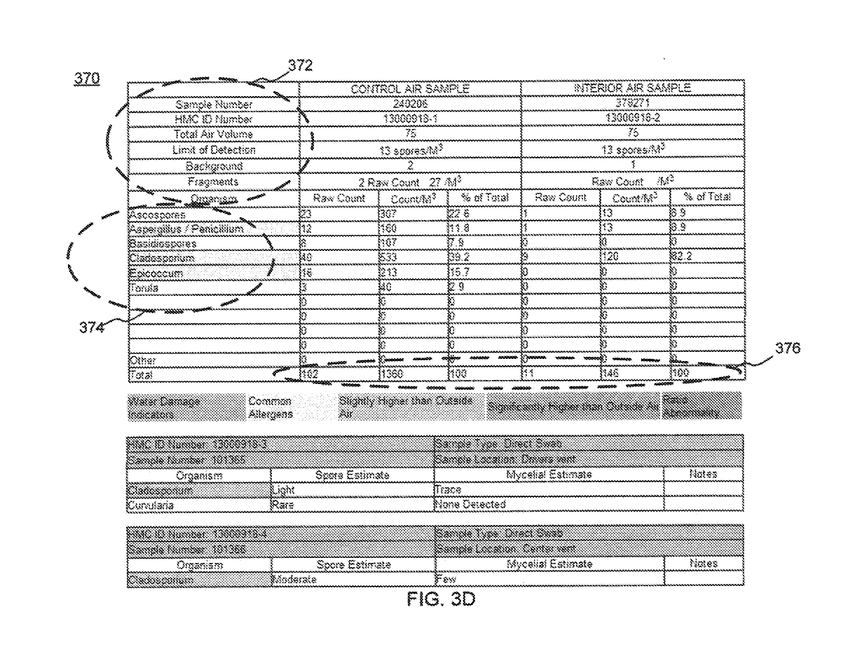

FIG. 3A illustrates a user interface 300 for inputting various sample rates, locations and other measurement parameters according to example embodiments. Referring to FIG. 3A, the interface 300 may include samples to be performed for laboratory analysis 310. The first sample may be a control air sample measured outside the vehicle to identify the particle or substance concentration in the air for control and comparison purposes. Next, a first interior air sample 314 may be setup to have corresponding measurement parameters. Additional samples may be setup, such as vent direct 316 samples, normal air inside vehicle, etc., until all desired test points have been taken into consideration for measurement purposes.

FIG. 3B illustrates another user interface 350 used to setup a moisture screening procedure 352 in addition to the air particle measurement procedure in FIG. 3A. The report section 352 may include additional menu options, drop-down menus and other items that can be selected prior to conducting the measurements. The various data selected and identified in the menu setup options of FIGS. 3A and 3B may be auto-populated into the final report along with other test results, vehicle images, warnings, color coded warnings (e.g., green “ok”, yellow “caution”, red “health hazard”, etc.).

FIG. 3C illustrates a test results page generated via the computer application after the results have been collected and processed for customer reporting purposes, according to example embodiments. Referring to FIG. 3C, the sample summary reports 360 includes four separate tests that were conducted during the test. The outdoor air control test 362 is identified for example purposes. Each test has its own numerical identifier which may be auto-generated by the application when the test is created. A sample volume, run time, and type are also associated with the test and may be set to default values which can be modified by user action or based on user input parameters which may invoke an automated test parameter modification. For example, if the user has identified various types of odors, visual mildew, etc., the test may need to modify its default parameters automatically to perform a different type of sensor feedback and processing function and/or modify the air volume amount, the test duration, etc., to accommodate a particular scenario identified at the onset of the test procedure.

FIG. 3D illustrates an example summary report 370 according to example embodiments. Referring to FIG. 3D, the report 370 may include various parameters for user analysis. The parameters may be generated by the automated application based on sensor readings and other input which may be digitized and processed by a computer processor operating in association with an operating system of the computing device. The report 370 may include various information items, such as a summary 372, a list of mold (mould)s/organisms 374 and their respective concentrations, and a list of total concentrations for all the mold (mould)s/organisms 376. Examples of such mold (mould)s/organisms, may include Ascospores, Aspergillus/Penicillium, Basidiospores, Cladosporium, Epicoccum, and Torula 374. A designation (e.g., light, moderate, heavy, etc.) may be selected based on predefined threshold levels of mold (mould)s/organisms identified from the sensor results and placed into the chart automatically based on the concentrations tested.

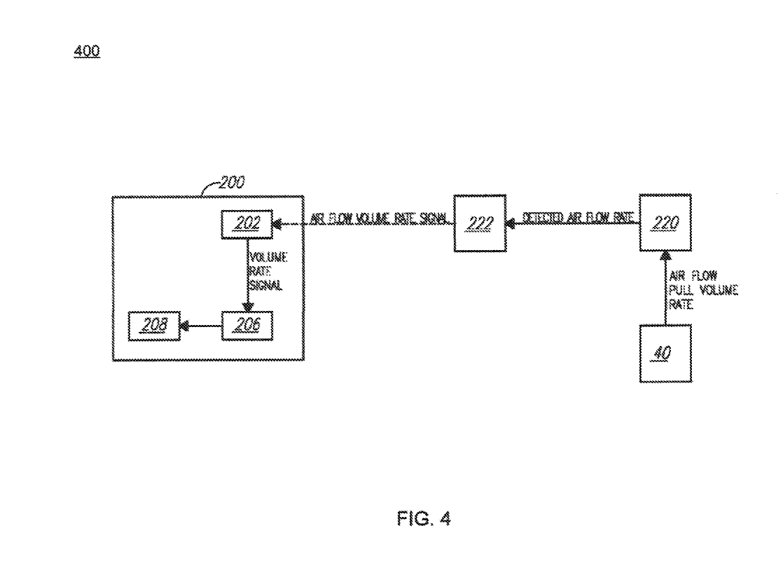

FIG. 4 illustrates a vacuum rate indicator 400 which includes a receiver 202 for wirelessly receiving signals transmitted via an air flow rate sensor 220. The air flow rate sensor 220 is suitably disposed along an inner circumferential sidewall of an inlet port of the vacuum source 40. The sensor 220 detects the air flow pull volume rate as air is pulled through the system via the vacuum source 40. The sensor 220 communicates detected air flow volume rate to a transceiver 222 being in connection therewith, the transceiver 222 transmits wirelessly an air flow volume rate signal received by a transceiver 202 in connection with indicator 200. Transceiver 202 of indicator 200 transmits the received air flow volume rate signal to an indicator drive 206 in connection with transceiver 202, the indicator drive 206 enables the air flow volume rate signal to be displayed on a display panel 208 as an air flow volume rate measure, e.g. 15 liters per minute. The display panel 208 may be a liquid crystal display (LCD) display panel; however, other display panels 208 are envisioned, such as a light-emitting diode (LED) display panel (not shown). The air flow volume rate is preferably displayed by display panel 208 as a measure of liters per minute; however, other units of measure are envisioned, such as feet per sec (fps).

A computer (not shown) may be coupled to the transceiver 202, the indicator drive 206 and/or the display panel 208 to receive and generate organism/mold (mould) concentration results based on the sensor readings received. The computer may be operated by a test application the receives the test results, aggregates the data, determines whether any thresholds have been met and/or exceeded and provides a summary of all mold (mould)/organism concentrations included in the air tests performed on the car. Any alerts necessary for the safety of the driver of the vehicle may be displayed in a green, yellow, red format so the user can observe the risks of being in the vehicle. If a particular mold (mould) or organism is found to be too high a concentration based on predefined levels of air concentration, then the application may provide a safety indicator alerting the user if necessary in the report. To the contrary, if all levels are optimal, then a report may be generated as having no alerting colors or indications so the user may not be alarmed about the present air quality inside their vehicle.

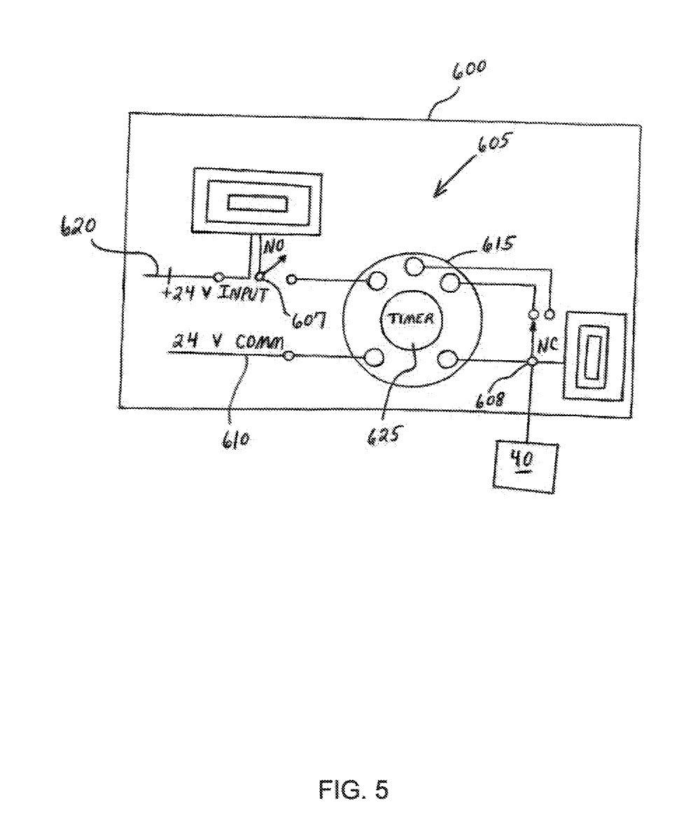

Referring now to FIG. 5, another alternate embodiment is disclosed wherein the system of the present application includes a timer mechanism 600, electrically coupled to the vacuum source 40, which is provided for automatically controlling activation and deactivation of the vacuum source 40 over predetermined time interval(s). For purposes of this disclosure, “activation” of vacuum source is intended to mean the powering on or more particularly, the actuation of vacuum source 40 suction function/operation, and “deactivation” is intended to mean actuation of vacuum source 40 to an off mode, state, or condition. The timer mechanism 600 comprises a timing circuit 605 which includes a normally open (NO) contact 607, a normally closed (NC) contact 608, a common terminal 610 in connection with NC contact 608, the NC contact 608 coupled and outputting to the vacuum source 40, and a time delay relay 615. The time delay relay 615 may be defined as a Single Pole Double Throw (SPDT) relay. The time delay relay 615 is coupled to a power supply 620 having sufficient voltage (shown herein as +24 volts) to enable activation of the time delay relay 615. The application of voltage from the power supply 620 starts an adjustable or programmable timer 625 set for a predetermined first interval, or period of delay. According to one embodiment, the programmable timer 625 is set for approximately 3 minutes. After the first interval has been exceeded, the time delay relay 615 will trip automatically, sending an appropriate signal as input to the vacuum source 40 to cause activation of vacuum source 40, the vacuum source 40 maintains 5 activation for a predetermined second interval via application of voltage from power supply 620 actuating programmable timer 625 set for the predetermined second interval, e.g., 5 minutes.

More specifically, voltage applied from power supply 620 activates closure of the NO contact 607 (“flexed” position) and opening of the NC contact 608 (“flexed” position). Closure of NO contact 607 allows current to pass from power supply 620 and through NO contact 607 to energize the timer 625 which activates the predetermined first interval. While the NC contact 608 is in an open position, the passage of current to the vacuum source 40 is prevented, thereby temporarily maintaining vacuum source 40 in a de-energized or deactivated mode. After the first interval expires or has been exceeded, time delay relay 615 trips automatically sending an appropriate signal as input to the vacuum source 40 to cause activation thereof, and NO contact 607 opens (“resting” position), and NC contact 608 closes (“resting” position). While the NC contact 608 is in a closed position, current passes through the relay circuit 615 and to the vacuum source 40, thus activating or energizing vacuum source 40. While the NO contact 607 is in an open position, the passage of current to programmable timer 625 is prevented, thereby placing programmable timer 625 in a de-energized mode. After the second interval expires or has been exceeded, the NC contact 608 automatically opens, thus placing vacuum source 40 in a deactivated mode.

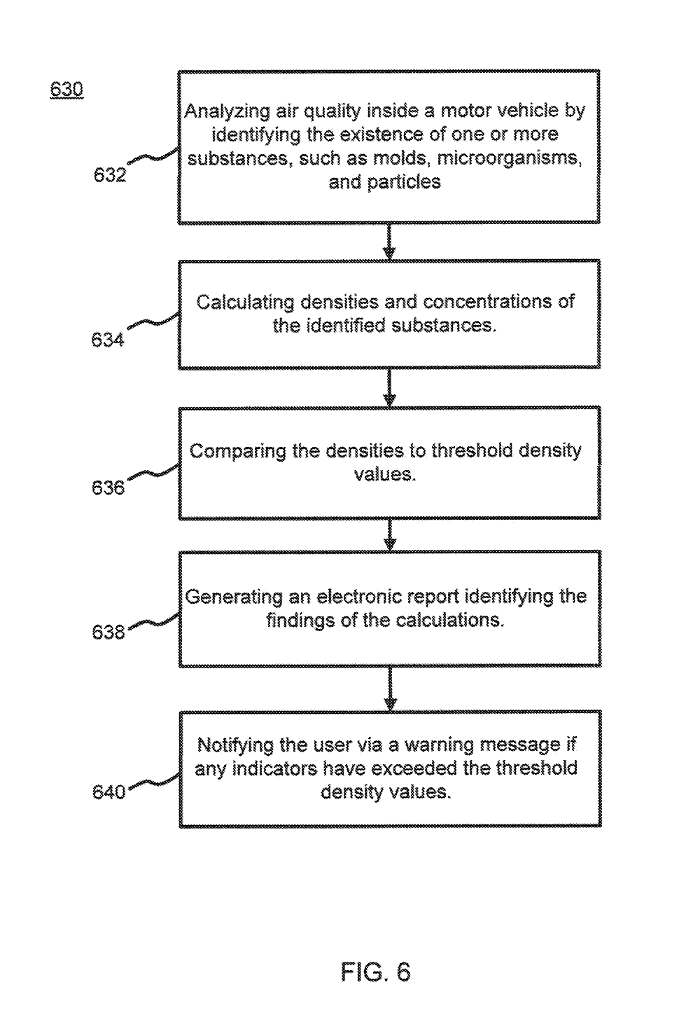

FIG. 6 illustrates an example method of operation of the screening procedure. Referring to FIG. 6, the flow diagram 630 includes various operations included in the screening procedure. For example, for analysing air quality inside a motor vehicle, the system may perform identifying the existence of one or more substances at operation 632, such as mold (mould)s, microorganisms, and particles. Calculating densities of the identified substances at operation 634, comparing the densities to threshold density values at operation 636 and generating an electronic report identifying the findings of the calculations at operation 638 and notify the user via a warning message if any indicators have exceeded the threshold density values at operation 640.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

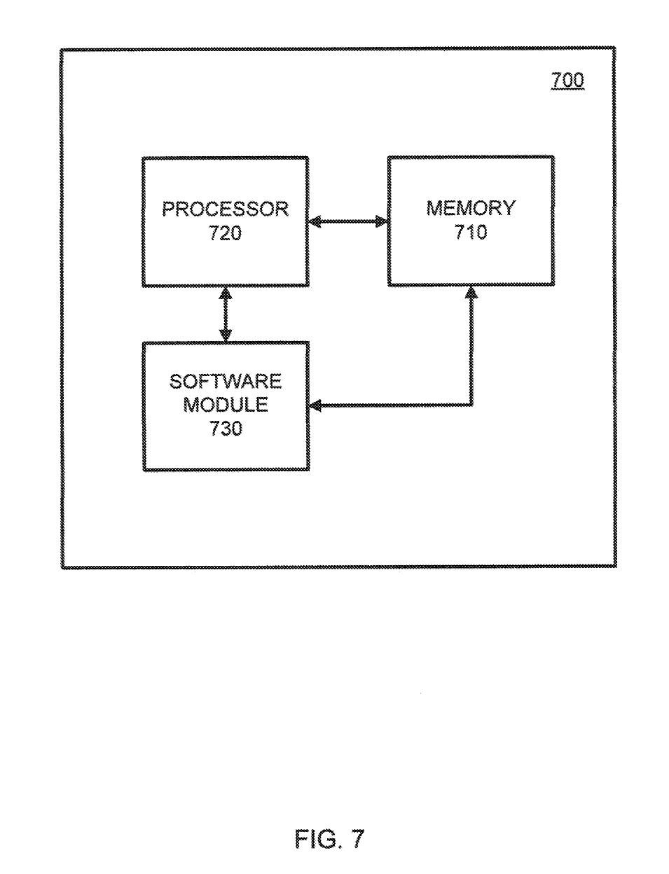

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example FIG. 7 illustrates an example network element 700, which may represent any of the above-described network components, etc.

As illustrated in FIG. 7, a memory 710 and a processor 720 may be discrete components of the network entity 700 that are used to execute an application or set of operations. The application may be coded in software in a computer language understood by the processor 720, and stored in a computer readable medium, such as, the memory 710. The computer readable medium may be a non-transitory computer readable medium that includes tangible hardware components in addition to software stored in memory. Furthermore, a software module 730 may be another discrete entity that is part of the network entity 700, and which contains software instructions that may be executed by the processor 720. In addition to the above noted components of the network entity 700, the network entity 700 may also have a transmitter and receiver pair configured to receive and transmit communication signals (not shown).

Although an exemplary embodiment of the system, method, and computer readable medium of the present invention has been illustrated in the accompanied drawings and described in the foregoing detailed description, it will be understood that the invention is not limited to the embodiments disclosed, but is capable of numerous rearrangements, modifications, and substitutions without departing from the spirit or scope of the invention as set forth and defined by the following claims. For example, the capabilities of the system can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present invention in any way, but is intended to provide one example of many embodiments of the present invention. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the invention, as generally described and illustrated in the figures herein, may be arranged and designed in a wide variety of different configurations. Thus, the detailed description of the embodiments is not intended to limit the scope of the invention as claimed, but is merely representative of selected embodiments of the invention.

One having ordinary skill in the art will readily understand that the invention as discussed above may be practiced with steps in a different order, and/or with hardware elements in configurations that are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method of analysing air quality inside a motor vehicle, the method comprising:
conducting an air sampling procedure of air inside the motor vehicle;
identifying an existence of one or more substances within that air sampled inside the motor vehicle via one or more sensors;
calculating densities of the one or more identified substances via a processor;
comparing the densities to corresponding threshold density values stored in memory; and
generating an electronic report identifying the findings of the calculations.

2. The method of claim 1, further comprising:
generating a notification parameter on the electronic report to identify the user if any indicators have exceeded the threshold density values.

3. The method of claim 1, wherein the one or more substances comprise at least one of mold (mould)s, microorganisms, and particles.
